(19) United States
(12) Patent Application Publication
(10) Pub. No.: US 2019/0309248 A1
(43) Pub. Date: Oct. 10, 2019

(54) Novel Fermentation Systems and Methods

(71) Applicant: Locus IP Company, LLC, Solon, OH (US)

(72) Inventors: Ken ALIBEK, Solon, OH (US); Sean FARMER, North Miami Beach, FL (US); Kent ADAMS, Twinsburg, OH (US)

(21) Appl. No.: 16/470,224

(22) PCT Filed: Jan. 5, 2018
(86) PCT No.: PCT/US2018/012561

§ 371 Date: Jun. 16, 2019

Related U.S. Application Data
(60) Provisional application No. 62/443,356, filed on Jan. 6, 2017.

(51) Int. Cl. C12M 1/00; C12N 1/16; C12M 1/12; C12P 19/44; A01N 63/04; C09K 8/584; C09K 8/52; C11D 1/66; C11D 3/38; C05F 11/08; A61L 2/22; A61L 2/07; A61L 2/10; B08B 9/027; C02F 1/00; C02F 1/32; A23K 10/18; A01C 21/00; E21B 43/16

(52) U.S. Cl. CPC C12M 23/58; C02F 2303/04; C12M 29/14; C12N 1/16; C12M 37/00; C12P 19/44; A01N 63/04; C09K 8/584; C09K 8/52; C11D 1/662; C11D 3/381; C05F 11/08; A61L 2/22; A61L 2/07; A61L 2/10; B08B 9/027; C02F 1/001; C02F 1/32; A23K 10/18; A01C 21/00; E21B 43/16; A61L 2202/17; B08B 2209/027; C12M 23/52

(57) ABSTRACT

The subject invention provides systems and apparatuses for producing microbe-based compositions that can be used in the oil and gas industry, environmental cleanup, as well as for other applications. More specifically, the present invention includes biological reactors, equipment, and materials for fermenting microbe-based compositions.

### CROSS-REFERENCE TO A RELATED APPLICATION

This application claims the benefit of U.S. provisional application Ser. No. 62/443,356, filed Jan. 6, 2017, which is incorporated herein by reference in its entirety.

### FIELD OF THE INVENTION

The present invention relates to methods and systems for producing microbe-based compositions that can be used in, for example, the oil industry, agriculture, mining, waste treatment and bioremediation.

### BACKGROUND OF THE INVENTION

Cultivation of microorganisms such as bacteria, yeast and fungi is important for the production of a wide variety of useful bio-preparations. Microorganisms play crucial roles in, for example, food industries, pharmaceuticals, agriculture, mining, environmental remediation, and waste management.

There exists an enormous potential for the use of microbes in a broad range of industries. The restricting factor in commercialization of microbe-based products has been the cost per propagule density, where it is particularly expensive and unfeasible to apply microbial products to large scale operations with sufficient inoculum to see the benefits.

Two principle forms of microbe cultivation exist: submerged cultivation and surface cultivation. Bacteria, yeasts and fungi can all be grown using either the surface or submerged cultivation methods. Both cultivation methods require a nutrient medium for the growth of the microorganisms. The nutrient medium, which can either be in a liquid or a solid form, typically includes a carbon source, a nitrogen source, salts and appropriate additional nutrients and microelements. The pH and oxygen levels are maintained at values suitable for a given microorganism.

Microbes have the potential to play highly beneficial roles in, for example, the oil and agriculture industries, if only they could be made more readily available and, preferably, in a more active form.

Oil and natural gas are obtained by drilling into the earth's surface using what is generically referred to as a drilling rig. A well or borehole begins by drilling a large diameter hole (e.g., 24-36 inches yin diameter) into the ground using a drill bit. The drill bit is attached to a drill pipe, which is rotated by the drilling rig. The drilling rig generally continues to drill a large hole until the drill bit passes beneath the water table. Next, a metal liner (or casing) is placed in the large diameter hole and cement is pumped through the inside of the liner. When the cement reaches the bottom of the liner, it flows upward, filling the void between the liner and the surrounding formation, isolating the water table and protecting it from whatever drilling fluids are pumped down the hole in subsequent steps.

After the first casing is cemented in, a medium sized bit can be used to drill deeper into the subterranean formation. There are generally one or more stopping points where the drill bit is removed, followed by a smaller casing liner and cement. This process is repeated until the well is completed.

During the drilling process, drilling fluids are pumped through the drill pipe and out of the drill bit. This fluid then flows back up in the space between the drill pipe and the formation or casing. The drilling fluid removes drill cuttings, balances downhole pressures, lubricates the borehole, and also works to clean the borehole of friction-causing substances.

After the well is drilled, a production liner (or casing) is generally set and the well is then perforated (e.g., explosives are used to puncture the production liner at specific points in the oil bearing formation). Oil then begins to flow out of the well, either under the natural pressure of the formation or by using pressure that is induced via mechanical equipment, water flooding, or other means. As the crude oil flows through the well, substances in the crude oil often collect on the surfaces of the production liners, causing reduction in flow, and sometimes even stopping production all together.

A variety of different chemicals and equipment are utilized to prevent and remediate this issue, but there is a need for improved products and methods. In particular, there is a need for products and methods that are more environmentally friendly, less toxic, and have improved effectiveness.

In the agriculture industry, farmers have relied heavily on the use of synthetic chemicals and chemical fertilizers to boost yields and protect crops against pathogens, pests, and disease; however, when overused or improperly applied, these substances can be air and water pollutants through runoff, leaching and evaporation. Even when properly used, the over-dependence and long-term use of certain chemical fertilizers and pesticides deleteriously alters soil ecosystems, reduces stress tolerance, increases pest resistance, and impedes plant and animal growth and vitality.

Mounting regulatory mandates governing the availability and use of chemicals, and consumer demands for residue free, sustainably-grown food produced with minimal harm to the environment, are impacting the industry and causing an evolution of thought regarding how to address the myriad of challenges. The demand for safer pesticides and alternate pest control strategies is increasing. While wholesale elimination of chemicals is not feasible at this time, farmers are increasingly embracing the use of biological measures as viable components of Integrated Nutrient Management and Integrated Pest Management programs.

For example, in recent years, biological control of nematodes has caught great interest. This method utilizes biological agents as pesticides, such as live microbes, bio-products derived from these microbes, and combinations thereof. These biological pesticides have important advantages over other conventional pesticides. For example, they are less harmful compared to the conventional chemical pesticides. They are more efficient and specific. They often biodegrade quickly, leading to less environmental pollution.

The use of biopesticides and other biological agents has been greatly limited by difficulties in production, transportation, administration, pricing and efficacy. For example, many microbes are difficult to grow and subsequently deploy to agricultural and forestry production systems in sufficient quantities to be useful. This problem is exacerbated by losses in viability and/or activity due to processing, formulating, storage, and stabilizing prior to distribution. Furthermore, once applied, biological products may not thrive for any number of reasons including, for example, insufficient initial cell densities, the inability to compete effectively with the existing microflora at a particular location, and being introduced to soil and/or other environmental conditions in which the microbe cannot flourish or even survive.

Microbe-based compositions could help resolve some of the aforementioned issues faced by the agriculture industry, the oil and gas industry, as well as many others. Thus, there is a need for more efficient cultivation methods for mass production of microorganisms and microbial metabolites.

### BRIEF SUMMARY OF THE INVENTION

The present invention provides materials, methods and systems for producing microbe-based compositions that can be used in the oil and gas industry, agriculture, health care and environmental cleanup, as well as for a variety of other applications. Specifically, the subject invention provides materials, methods and systems for efficient cultivation of microorganisms and production of microbial growth by-products.

Embodiments of the present invention provide novel, low-cost fermentation methods and systems. More specifically, the present invention provides biological reactors for fermentation. In specific embodiments, the systems are used to grow yeast- and/or other microbe-based compositions. In certain specific embodiments, the systems can be used for the production of Starmerella bombicola yeast compositions.

The systems can be used to grow yeast, fungi and bacteria. In certain embodiments, the systems can be used for the production of yeast-based compositions, including, for example, compositions comprising Starmerella bombicola, Wickerhamomyces anomalus, and/or Pseudozyma aphidis yeast. In some embodiments, the systems can be used for the production of bacteria-based compositions, including, for example, compositions comprising Bacillus subtilis and/or Bacillus licheniformis.

In a specific embodiment, the system of the subject invention comprises at least two tanks that are connected to each other by tubing. In this multi-tank reactor, a pump forces microbial culture through the tubing from one tank to another tank. In preferred embodiments, the tubing is installed at, or near, the top of the tanks. While the culture is moving through the tubing, it can be oxygenated by air pushed into the fluid stream by, for example, an air compressor. This mixes and oxygenates the culture. Closer to the bottom of the tanks, another tube connects the two tanks in order to balance the culture levels in each tank. This tubing can have another entry to facilitate air supplementation. This tubing can, therefore, provide additional mixing and aeration. Additionally, both tanks can be supplemented with individual sparging systems.

Inoculation can take place in one or both of the tanks and the inoculum is mixed in both tanks through the aforementioned tubing systems. In preferred embodiments of the multi-tank system, the pump or pumps operate continuously throughout the process of fermentation. The flow rate can be, for example, from 10 to 20 to 200 gallons per minute. In specific embodiments, a full culture exchange occurs between the tanks every 5 to 10 minutes.

Advantageously, the systems of the present invention can be scaled depending on the intended use. For example, the tanks can range in size from a few gallons to tens of thousands of gallons.

In one embodiment, the subject invention provides methods of cultivating microorganisms without contamination using the subject system. In certain embodiments, the methods of cultivation comprise adding a culture medium comprising water and nutrient components to the subject systems using, for example, a peristaltic pump; inoculating the system with a viable microorganism; and optionally, adding an antimicrobial agent to the culture medium. The antimicrobial agent can be, for example, an antibiotic or a sophorolipid.

In one embodiment, the subject invention further provides a composition comprising at least one type of microorganism and/or at least one microbial metabolite produced by the microorganism that has been grown using the fermentation system of the subject invention. The microorganisms in the composition may be in an active or inactive form. The composition may also be in a dried form or a liquid form.

Advantageously, the method and equipment of the subject invention reduce the capital and labor costs of producing microorganisms and their metabolites on a large scale. Furthermore, the cultivation process of the subject invention reduces or eliminates the need to concentrate organisms after completing cultivation. The subject invention provides a cultivation method that not only substantially increases the yield of microbial products per unit of nutrient medium but simplifies production and facilitates portability.

Portability can result in significant cost savings as microbe-based compositions can be produced at, or near, the site of intended use. This means that the final composition can be manufactured on-site using locally-sourced materials if desired, thereby reducing shipping costs. Furthermore, the compositions can include viable microbes at the time of application, which can increase product effectiveness.

Thus, in certain embodiments, the systems of the subject invention harness the power of naturally-occurring local microorganisms and their metabolic by-products. Use of local microbial populations can be advantageous in settings including, but not limited to, environmental remediation (such as in the case of an oil spill), animal husbandry, aquaculture, forestry, pasture management, turf management, horticultural ornamental production, waste disposal and treatment, mining, oil recovery, and human health, including in remote locations.

Compositions produced by the present invention can also be used in a wide variety of petroleum industry applications, such as microbially enhanced oil recovery. These applications include, but are not limited to, enhancement of crude oil recovery; stimulation of oil and gas wells (to improve the flow of oil into the well bore); removal of contaminants and/or obstructions such as paraffins, asphaltenes and scale from equipment such as rods, tubing, liners, tanks and pumps; prevention of the corrosion of oil and gas production and transportation equipment; reduction of H₂S concentration in crude oil and natural gas; reduction in viscosity of crude oil; upgradation of heavy crude oils and asphaltenes into lighter hydrocarbon fractions; cleaning of tanks, flowlines and pipelines; enhancing the mobility of oil during water flooding though selective and non-selective plugging; and fracturing fluids.

When used in oil and gas applications, the systems of the present invention can be used to lower the cost of microbial-based oilfield compositions and can be used in combination with other chemical enhancers, such as polymers, solvents, fracking sand and beads, emulsifiers, surfactants, and other materials known in the art.

### BRIEF DESCRIPTION OF THE FIGURE

FIG. 1 shows a two-tank system according to one embodiment of the invention.

FIG. 2 shows a side view of a two-tank system according to one embodiment of the invention, including exemplary tank measurements.

### DETAILED DESCRIPTION OF THE INVENTION

The present invention provides materials, methods and systems for producing microbe-based compositions that can be used in the oil and gas industry, aquaculture, agriculture, environmental cleanup, human health, as well as other applications. More specifically, in preferred embodiments the present invention provides biological reactors for fermenting yeast-based and/or other microbe-based compositions.

Embodiments of the present invention also provide novel, low-cost fermentation methods and systems. The systems can be used to cultivate yeast, fungi and bacteria and/or their growth by-products. In certain embodiments, the systems can be used for the production of yeast-based compositions, including, for example, compositions comprising Starmerella bombicola, Wickerhamomyces anomalus, and/or Pseudozyma aphidis yeast. In some embodiments, the systems can be used for the production of bacteria-based compositions, including, for example, compositions comprising Bacillus subtilis and/or Bacillus licheniformis.

In a preferred embodiment wherein yeasts are cultured, the resulting composition can have one or more of the following advantageous properties: high concentrations of mannoprotein and beta-glucan as part of the yeasts' cell wall; and the presence of biosurfactants and other microbial metabolites (e.g., lactic acid and ethanol, etc.) in the culture.

### Selected Definitions

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. In preferred embodiments, the microbes are present, with broth in which they were grown, in the microbe-based composition. The cells may be present at, for example, a concentration of 1×10⁴, 1×10⁵, 1×10⁶, 1×10⁷, 1×10⁸, 1×10⁹, 1×10¹⁰, or 1×10¹¹ or more propagules per milliliter of the composition. As used herein, a propagule is any portion of a microorganism from which a new and/or mature organism can develop, including but not limited to, cells, spores, mycelia, buds and seeds.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers, such as plant hormones, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “harvested” refers to removing some or all of the microbe-based composition from a growth vessel.

As used herein, a “biofilm” is a complex aggregate of microorganisms, such as bacteria, wherein the cells adhere to each other. The cells in biofilms are physiologically distinct from planktonic cells of the same organism, which are single cells that can float or swim in liquid medium.

As used herein, the term “control” used in reference to the activity produced by the subject microorganisms extends to the act of killing, disabling or immobilizing pests or otherwise rendering the pests substantially incapable of causing harm.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. As used herein, reference to “isolated” in the context of a microbial strain means that the strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state.

A “metabolite” refers to any substance produced by metabolism or a substance necessary for taking part in a particular metabolic process. A metabolite can be an organic compound that is a starting material (e.g., glucose), an intermediate (e.g., acetyl-CoA) in, or an end product (e.g., n-butanol) of metabolism. Examples of metabolites include, but are not limited to, enzymes, toxins, acids, solvents, alcohols, proteins, vitamins, minerals, microelements, amino acids, and biosurfactants.

As used herein, “surfactant” refers to a compound that lowers the surface tension (or interfacial tension) between two liquids or between a liquid and a solid. Surfactants act as detergents, wetting agents, emulsifiers, foaming agents, and dispersants. A “biosurfactant” is a surfactant produced by a living organism.

### Fermentation System Design and Operation

In a specific embodiment, the system of the subject invention comprises at least two tanks that are connected to each other by tubing. A pump forces microbial culture through the tubing from one tank to another tank. In preferred embodiments, the tubing is installed at, or near, the top of the tanks. The pump can have an input connected to the first tank via a first tube (or hose or pipe), and an output connected to the second tank via a second tube.

One or more air compressors can be included for aeration and each air compressor can, optionally, have an air filter for preventing contamination. The air compressors can be connected to one or more gas injectors, bubblers, and/or spargers. Gas injectors can be located in, for example, any and/or all of the tubes and/or tanks of the reactor. The bubblers and/or spargers can be located in any and/or all of the tanks. While the culture is moving through the tubing, it can be oxygenated by air pushed into the fluid stream by, for example, the air compressor. This mixes and oxygenates the culture.

Closer to the bottom of the tanks, a third tube (or hose or pipe) can be connected from the second tank to the first tank. The third tube allows for liquid to flow under hydrostatic pressure from the second tank to the first tank. This tubing connects the two tanks in order to balance the culture levels in each tank. This tubing can have another entry to facilitate air supplementation. This tubing can, therefore, provide additional mixing and aeration. The system can include a flow control valve on the output of the first pump suitable for controlling the first pump flow rate. The first pump can also be controlled using a variable frequency motor so that flow rates can be properly adjusted through changes in electric frequency.

The tubing near the top of the two tanks is preferably connected to each tank at a point that is in the top 50%, 40%, 30%, 25%, 20%, 15%, 10%, 5%, 2%, and 1% of the tank. The tubing nearer to the bottom of the two tanks is preferably connected to each tank at a point that is in the bottom 50%, 40%, 30%, 25%, 20%, 15%, 10%, 5%, 2%, and 1% of the tank.

The pump and/or pumps of the system can be sized to be suitable for establishing a recycle ratio (the volume pumped per hour/the total volume of reactor liquid) ranging from, for example, 30 to 0.10. The pump can be a centrifugal pump. The system can include one or more block valves (any generic valve used to stop flow) on the first tank and second tank inlets and outlets. A hose can be connected to the first pump (or a second pump connected to the reactor) to drain the reactor and pump the composition to its place of intended use. A nozzle can be located at the end of the hose and be suitable for spraying the composition.

In preferred embodiments of the system, the pump or pumps operate continuously throughout the process of fermentation. The flow rate can be, for example, from 10 to 20 to 200 gallons per minute. In specific embodiments, a full culture exchange occurs between the tanks every 5 to 10 minutes.

The system can include one or more sight glasses on, for example, any and/or all of the tubes and/or tanks for visual monitoring of the fermentation process. Furthermore, any and/or all of the tubes can have a check-valve for preventing backflow.

One or more vents (or pressure release valves (PSVs)) can be located on any and/or all of the tanks. The vents or PSVs can allow gases to flow out, but do not allow air in (e.g., the valve can open when the internal gas pressure of the reactor goes above 1.2 atm and can close when the internal gas pressure falls below 1.1 atm).

The tanks used according to the subject invention can be any fermenter or cultivation reactor for industrial use. These tanks may be, for example, made of glass, polymers, metals, metal alloys, and combinations thereof. The tanks may be, for example, from 5 liters to 2,000 liters or more. Typically, the vessels will be from 10 to 1,500 liters, and preferably are from 100 to 1,000 liters, and more preferably from 250 to 750 liters, from 300 to 600 liters, or from 400 to 550 liters.

Prior to microbe growth, the tanks may be disinfected or sterilized. In one embodiment, fermentation medium, air, and equipment used in the method and cultivation process are sterilized. The cultivation equipment such as the reactor/vessel may be separated from, but connected to, a sterilizing unit, e.g., an autoclave. The cultivation equipment may also have a sterilizing unit that sterilizes in situ before starting the inoculation, e.g., by using a steamer. The air can be sterilized by methods know in the art. For example, the ambient air can pass through at least one filter before supplemented into the vessel. In other embodiments, the medium may be pasteurized or optionally no heat at all added, where the use of low water activity and low pH may be exploited to control bacterial growth.

The system can be used as a batch reactor (as opposed to a continuous reactor). Advantageously, the system can be scaled depending on its intended use. For small applications, such as, for example, bioremediation, the system can be as small as 50 gallons or even smaller. For applications where large volumes of the composition are necessary, such as microbially enhanced oil recovery, the system can be scaled to produce 20,000 or more gallons of product.

The system can include temperature controls. The system can be insulated so the fermentation process can remain at appropriate temperatures in low temperature environments. Any of the insulating materials known in the art can be applied including fiberglass, silica aerogel, ceramic fiber insulation, etc. The insulation can surround any and/or all of the tubes and/or tanks of the system.

The system can also be adapted to ensure maintaining an appropriate fermentation temperature. For example, the outside of the system can be reflective to avoid raising the system temperature during the day. Furthermore, a cooling system can be added that includes, for example, one or more of a cooling jacket and a cooling heat exchanger. The cooling water can exchange heat with ambient air and be recirculated through the cooling system. The heat exchanger and/or cooling jacket can surround or be installed within any and/or all of the tubes and/or tanks of the system. For extreme environments, the system can include refrigeration and cooling coils within the reactor, a jacket surrounding the reactor, or heat exchangers connected to the tubes.

The system can utilize an electric heater. However, for larger applications where heat is required, steam or hydrocarbon fuel can be utilized. A steam input and/or a steam source can be connected to one or more of a steam injector, a steam jacket, and a steam heat exchanger. The steam jacket can surround any and/or all of the tanks of the system. In addition, steam can be directly injected into any and/or all of the tubes and/or tanks of the system. A steam heat exchanger can be placed inside the reactor, steam can condense within the tubes of the heater exchanger, and then be expelled. The steam heat exchanger can be a closed system that does not mix water or steam into the reactor.

In one embodiment, the tanks have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

A thermometer can be included and the thermometer can be manual or automatic. The thermometer can preferably be placed on any and/or all of the tanks of the reactor. An automatic thermometer can manage the heat and cooling sources appropriately to control the temperature throughout the fermentation process. The desired temperatures can be programmed on-site or pre-programmed before the system is delivered to the fermentation site. The temperature measurements can then be used to automatically control the heating and cooling systems that are discussed above.

The pH adjustment can be accomplished by automatic means or it can be done manually. The automatic pH adjustment can include a pH probe and an electronic device to dispense pH adjustment substances appropriately, depending on the pH measurements. The pH probe is preferably placed on any and/or all of the tanks of the reactor. The pH can be set to a specific number by a user or can be pre-programmed to change the pH accordingly throughout the fermentation process. If the pH adjustment is to be done manually, pH measurement tools known in the art can be included with the system for manual testing.

A computer system for measuring and adjusting of pH and temperature can be used to monitor and control fermentation parameters for each tank of the reactors. The computer can be connected to a thermometer and a pH probe, for example. In addition to monitoring and controlling temperature and pH, each vessel may also have the capability for monitoring and controlling, for example, dissolved oxygen, agitation, foaming, purity of microbial cultures, production of desired metabolites and the like. The systems can further be adapted for remote monitoring of these parameters, for example with a tablet, smart phone, or other mobile computing device capable of sending and receiving data wirelessly.

In a further embodiment, the tanks may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of bacteria in a sample. The technique can also provide an index by which different environments or treatments can be compared.

In one embodiment, the fermentation system is a mobile or portable bioreactor that may be provided for on-site production of a microbiological product including a suitable amount of a desired strain of microorganism. Because the microbiological product is generated on-site of the application, without resort to the bacterial stabilization, preservation, storage and transportation processes of conventional production, a much higher density of live microorganisms may be generated, thereby requiring a much smaller volume of the microorganism composition for use in the on-site application. This allows for a scaled-down bioreactor (e.g., smaller fermentation tanks, smaller supplies of starter material, nutrients, pH control agents, and de-foaming agent, etc.) that facilitates the mobility and portability of the system.

The system can include a frame for supporting the apparatus components (including the tanks, flow loops, pumps, etc.). The system can include wheels for moving the apparatus, as well as handles for steering, pushing and pulling when maneuvering the apparatus.

The system can be configured on the back of one or more truck trailers and/or semi-trailers. That is, the system can be designed to be portable (i.e., the system can be suitable for being transported on a pickup truck, a flatbed trailer, or a semi-trailer).

### Microorganisms

The microorganisms grown according to the systems and methods of the subject invention can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. Procedures for making mutants are well known in the microbiological art. For example, ultraviolet light and nitrosoguanidine are used extensively toward this end.

The microbes and their growth products produced according to the subject invention can be used to produce a vast array of useful products, including, for example, biopesticides, biosurfactants, ethanol, nutritional compounds, therapeutic proteins such as insulin, compounds useful as vaccines, and other biopolymers. The microbes used as these microbial factories may be natural, mutated or recombinant.

In one embodiment, the microorganism is a yeast or fungus. Yeast and fungus species suitable for use according to the current invention, include Candida, Saccharomyces (S. cerevisiae, S. boulardii sequela, S. torula), Issalchenkia, Kluyveromyces, Pichia, Wickerhamomyces (e.g., W. anomalus), Starmerella (e.g., S. bombicola), Mycorrhiza, Mortierella, Phycomyces, Blakeslea, Thraustochytrium, Phythium, Entomophthora, Aureobasidium pullulans, Pseudozyma aphidis, Fusarium venenalum, Aspergillus, Trichoderma (e.g., T. reesei, T. harzianum, T. hamatum, T. viride), Rhizopus spp., Mycorrhiza (e.g., Glomus spp., Acaulospora spp., vesicular-arbuscular mycorrhizae (VAM), arbuscular mycorrhizae (AM)), endophytic fungi (e.g., Piriformis indica), any strain of killer yeastm, and combinations thereof.

In one embodiment, the yeast is a killer yeast. As used herein, “killer yeast” means a strain of yeast characterized by its secretion of toxic proteins or glycoproteins, to which the strain itself is immune. The exotoxins secreted by killer yeasts are capable of killing other strains of yeast, fungi, or bacteria. For example, microorganisms that can be controlled by killer yeast include Fusarium and other filamentous fungi. Examples of killer yeasts according to the present invention are those that can be used safely in the food and fermentation industries, e.g., beer, wine, and bread making; those that can be used to control other microorganisms that might contaminate such production processes; those that can be used in biocontrol for food preservation; those than can be used for treatment of fungal infections in both humans and plants; and those that can be used in recombinant DNA technology. Such yeasts can include, but are not limited to, Wickerhamomyces, Pichia (e.g., P. anomala, P. guielliermondii, P. kudriavzevii), Hansenula, Saccharomyces, Hanseniaspora, (e.g., H. uvarum), Ustilago maydis, Debaryomyces hansenii, Candida, Cryptococcus, Kluyveromyces, Torulopsis, Ustilago, Williopsis, Zygosaccharomyces (e.g., Z. bailii), and others.

In one embodiment, the microbe is a killer yeast, such as a Pichia yeast selected from Pichia anomala (Wickerhamomyces anomalus), Pichia guielliermondii, and Pichia kudriavzevii. Pichia anomala, in particular, is an effective producer of various solvents, enzymes, killer toxins, as well as sophorolipid biosurfactants.

In one embodiment, the microbial strain is chosen from the Starmerella clade. A culture of a Starmerella microbe useful according to the subject invention, Starmerella bombicola, can be obtained from the American Type Culture Collection (ATCC), 10801 University Blvd., Manassas, Va. 20110-2209 USA. The deposit has been assigned accession number ATCC No. 22214 by the depository.

The system can also utilize one or more strains of yeast capable of enhancing oil recovery and performing paraffin degradation, e.g., Starmerella (Candida) bombicola, Candida apicola, Candida batistae, Candida floricola, Candida riodocensis, Candida stellate, Candida kuoi, Candida sp. NRRL Y-27208, Rhodotorula bogoriensis sp., Wickerhamiella domericqiae, as well as any other sophorolipid-producing strains of the Starmerella clade. In a specific embodiment, the yeast strain is ATCC 22214 and mutants thereof.

In one embodiment, the microbe is a strain of Pseudozyma aphidis. This microbe is an effective producer of mannosylerythritol lipid biosurfactants.

In one embodiment, the microorganisms are bacteria, including gram-positive and gram-negative bacteria. These bacteria may be, but are not limited to, for example, Bacillus (e.g., B. subtilis, B. licheniformis, B. firmus, B. laterosporus, B. megaterium, B. amyloliquifaciens), Clostridium (C. butyricum, C. tyrobutyricum, C. acetobutyricum, Clostridium NIPER 7, and C. beijerinckii), Azobacter (A. vinelandii, A. chroococcum), Pseudomonas (P. chlororaphis subsp. aureofaciens (Kluyver), P. aeruginosa), Azospirillum brasiliensis, Ralslonia eulropha, Rhodospirillum rubrum, Sphingomonas (e.g., S. paucimobilis), Streptomyces (e.g., S. griseochromogenes, S. qriseus, S. cacaoi, S. aureus, and S. kasugaenis), Streptoverticillium (e.g., S. rimofaciens), Ralslonia (e.g., R. eulropha), Rhodospirillum (e.g., R. rubrum), Xanthomonas (e.g., X. campestris), Erwinia (e.g., E. carotovora), Escherichia coli, Rhizobium (e.g., R. japonicum, Sinorhizobium meliloti, Sinorhizobium fredii, R. leguminosarum biovar trifolii, and R. etli), Bradyrhizobium (e.g., B. japanicum, and B. parasponia), Arthrobacter (e.g., A. radiobacter), Azomonas, Derxia, Beijerinckia, Nocardia, Klebsiella, Clavibacter (e.g., C. xyli subsp. xyli and C. xyli subsp. cynodontis), Cyanobacteria, Pantoea (e.g., P. agglomerans), and combinations thereof.

In one embodiment, the microorganism is a strain of B. subtilis, such as, for example, B. subtilis var. lotuses B1 or B2, which are effective producers of, for example, surfactin and other biosurfactants, as well as biopolymers. This specification incorporates by reference International Publication No. WO 2017/044953 A1 to the extent it is consistent with the teachings disclosed herein. In another embodiment, the microorganism is a strain of Bacillus licheniformis, which is an effective producer of biosurfactants as well as biopolymers, such as levan.

In one embodiment, the microbe is a non-pathogenic strain of Pseudomonas. Preferably, the strain is a producer of rhamnolipid biosurfactants.

Other microbial strains including, for example, strains capable of accumulating significant amounts of, for example, glycolipid-biosurfactants, can be used in accordance with the subject invention. Other microbial by-products useful according to the present invention include mannoprotein, beta-glucan and other metabolites that have bio-emulsifying and surface/interfacial tension-reducing properties.

In one embodiment, a single type of microbe is grown in a vessel. In alternative embodiments, multiple microbes, which can be grown together without deleterious effects on growth or the resulting product, can be grown in a single vessel. There may be, for example, 2 to 3 or more different microbes grown in a single vessel at the same time.

### Methods of Cultivation Using the Subject Fermentation Systems

The subject invention provides methods and systems for the efficient production of microbes using novel biological reactors. The system can include all of the materials necessary for the fermentation (or cultivation) process, including, for example, equipment, sterilization supplies, and culture medium components, although it is expected that freshwater could be supplied from a local source and sterilized according to the subject methods.

In one embodiment, the system is provided with an inoculum of viable microbes. Preferably, the microbes are biochemical-producing microbes, capable of accumulating, for example, biosurfactants, enzymes, solvents, biopolymers, acids, and/or other useful metabolites. In particularly preferred embodiments, the microorganisms are biochemical-producing yeast (including killer yeasts), fungi, and/or bacteria, including without limitation those listed herein.

In one embodiment, the system is provided with a culture medium. The medium can include nutrient sources, for example, a carbon source, a lipid source, a nitrogen source, and/or a micronutrient source. Each of the carbon source, lipid source, nitrogen source, and/or micronutrient source can be provided in an individual package that can be added to the reactor at appropriate times during the fermentaton process. Each of the packages can include several sub-packages that can be added at specific points (e.g., when yeast, pH, and/or nutrient levels go above or below a specific concentration) or times (e.g., after 10 hours, 20 hours, 30 hours, 40 hours, etc.) during the fermentation process.

Before fermentation the tanks can be washed with a hydrogen peroxide solution (e.g., from 2.0% to 4.0% hydrogen peroxide; this can be done before or after a hot water rinse at, e.g., 80-90° C.) to prevent contamination. In addition, or in the alternative, the tanks can be washed with a commercial disinfectant, a bleach solution and/or a hot water or steam rinse. The system can come with concentrated forms of the bleach and hydrogen peroxide, which can later be diluted at the fermentation site before use. For example, the hydrogen peroxide can be provided in concentrated form and be diluted to formulate 2.0% to 4.0% hydrogen peroxide (by weight or volume) for pre-rinse decontamination.

In a specific embodiment, the method of cultivation comprises sterilizing the subject fermentation reactors prior to fermentation. The internal surfaces of the reactor (including, e.g., tanks, ports, spargers and mixing systems) can first be washed with a commercial disinfectant; then fogged (or sprayed with a highly dispersed spray system) with 2% to 4% hydrogen peroxide, preferably 3% hydrogen peroxide; and finally steamed with a portable steamer at a temperature of about 105° C. to about 110° C., or greater.

The culture medium components (e.g., the carbon source, water, lipid source, micronutrients, etc.) can also be sterilized. This can be achieved using temperature decontamination and/or hydrogen peroxide decontamination (potentially followed by neutralizing the hydrogen peroxide using an acid such as HCl, H₂SO₄, etc.).

In a specific embodiment, the water used in the culture medium is UV sterilized using an in-line UV water sterilizer and filtered using, for example, a 0.1-micron water filter. In another embodiment, all nutritional and other medium components can be autoclaved prior to fermentation.

To further prevent contamination, the culture medium of the system may comprise additional acids, antibiotics, and/or antimicrobials, added before, and/or during the cultivation process. The one or more antimicrobial substances can include, e.g., streptomycin, oxytetracycline, sophorolipids, and rhamnolipids.

Inoculation can take place in any and/or all of the reactor tanks, at which point the inoculum is mixed using through the tubing systems. Total fermentation times can range from 10 to 200 hours, preferably from 20 to 180 hours.

The fermenting temperature utilized in the subject systems and methods can be, for example, from about 25 to 40° C., although the process may operate outside of this range. In one embodiment, the method for cultivation of microorganisms is carried out at about 5° to about 100° C., preferably, 15° to 60° C., more preferably, 25 to 50° C. In a further embodiment, the cultivation may be carried out continuously at a constant temperature. In another embodiment, the cultivation may be subject to changing temperatures.

The pH of the medium should be suitable for the microorganism of interest. Buffering salts, and pH regulators, such as carbonates and phosphates, may be used to stabilize pH near an optimum value. When metal ions are present in high concentrations, use of a chelating agent in the liquid medium may be necessary.

In certain embodiments, the microorganisms can be fermented in a pH range from about 2.0 to about 10.0 and, more specifically, at a pH range of from about 3.0 to about 7.0 (by manually or automatically adjusting pH using bases, acids, and buffers; e.g., HCl, KOH, NaOH, H₂SO₄, and/or H₃PO₄). The invention can also be practiced outside of this pH range.

The fermentation can start at a first pH (e.g., a pH of 4.0 to 4.5) and later change to a second pH (e.g., a pH of 3.2-3.5) for the remainder of the process to help avoid contamination as well as to produce other desirable results (the first pH can be either higher or lower than the second pH). In some embodiments, pH is adjusted from a first pH to a second pH after a desired accumulation of biomass is achieved, for example, from 0 hours to 200 hours after the start of fermentation, more specifically from 12 to 120 hours after, more specifically from 24 to 72 hours after.

In one embodiment, the moisture level of the culture medium should be suitable for the microorganism of interest. In a further embodiment, the moisture level may range from 20% to 90%, preferably, from 30 to 80%, more preferably, from 40 to 60%.

The cultivation processes of the subject invention can be anaerobic, aerobic, or a combination thereof. Preferably, the process is aerobic, keeping the dissolved oxygen concentration above 10 or 15% of saturation during fermentation, but within 20% in some embodiments, or within 30% in some embodiments.

Advantageously, the system provides easy oxygenation of the growing culture with, for example, slow motion of air to remove low-oxygen containing air and introduction of oxygenated air. The oxygenated air may be ambient air supplemented periodically, such as daily.

Additionally, antifoaming agents can also be added to the system prevent the formation and/or accumulation of foam when gas is produced during cultivation and fermentation.

In one embodiment, the microbe-based composition does not need to be further processed after fermentation (e.g., yeast, metabolites, and remaining carbon sources do not need to be separated from the sophorolipids). The physical properties of the final product (e.g., viscosity, density, etc.) can also be adjusted using various chemicals and materials that are known in the art.

In one embodiment, the culture medium used in the subject system, may contain supplemental nutrients for the microorganism. Typically, these include carbon sources, proteins, fats, or lipids, nitrogen sources, trace elements, and/or growth factors (e.g., vitamins, pH regulators). It will be apparent to one of skill in the art that nutrient concentration, moisture content, pH, and the like may be modulated to optimize growth for a particular microbe.

The lipid source can include oils or fats of plant or animal origin which contain free fatty acids or their salts or their esters, including triglycerides. Examples of fatty acids include, but are not limited to, free and esterified fatty acids containing from 16 to 18 carbon atoms, hydrophobic carbon sources, palm oil, animal fats, coconut oil, oleic acid, soybean oil, sunflower oil, canola oil, stearic and palmitic acid.

The culture medium of the subject system can further comprise a carbon source. The carbon source is typically a carbohydrate, such as glucose, xylose, sucrose, lactose, fructose, trehalose, galactose, mannose, mannitol, sorbose, ribose, and maltose; organic acids such as acetic acid, fumaric acid, citric acid, propionic acid, malic acid, malonic acid, and pyruvic acid; alcohols such as ethanol, propanol, butanol, pentanol, hexanol, erythritol, isobutanol, xylitol, and glycerol; fats and oils such as canola oil, soybean oil, rice bran oil, olive oil, corn oil, sesame oil, and linseed oil; etc. Other carbon sources can include arbutin, raffinose, gluconate, citrate, molasses, hydrolyzed starch, potato extract, corn syrup, and hydrolyzed cellulosic material. The above carbon sources may be used independently or in a combination of two or more.

In one embodiment, growth factors and trace nutrients for microorganisms are included in the medium of the system. This is particularly preferred when growing microbes that are incapable of producing all of the vitamins they require. Inorganic nutrients, including trace elements such as iron, zinc, potassium, calcium copper, manganese, molybdenum and cobalt; phosphorous, such as from phosphates; and other growth stimulating components can be included in the culture medium of the subject systems. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included, e.g., L-Alanine.

In one embodiment, inorganic or mineral salts may also be included. Inorganic salts can be, for example, potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium carbonate. These inorganic salts may be used independently or in a combination of two or more.

The culture medium of the subject system can further comprise a nitrogen source. The nitrogen source can be, for example, in an inorganic form such as potassium nitrate, ammonium nitrate, ammonium sulfate, ammonium phosphate, ammonia, urea, and ammonium chloride, or an organic form such as proteins, amino acids, yeast extracts, yeast autolysates, corn peptone, casein hydrolysate, and soybean protein. These nitrogen sources may be used independently or in a combination of two or more.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

### Preparation of Microbe-Based Products

The microbe-based products of the subject invention include products comprising the microbes and/or microbial growth by-products and optionally, the growth medium and/or additional ingredients such as, for example, water, carriers, adjuvants, nutrients, viscosity modifiers, and other active agents.

One microbe-based product of the subject invention is simply the fermentation medium containing the microorganism and/or the microbial growth by-products produced by the microorganism and/or any residual nutrients. The product of feiinentation may be used directly without extraction or purification. If desired, extraction and purification can be easily achieved using standard extraction methods or techniques known to those skilled in the art.

The microorganisms in the microbe-based products may be in an active or inactive form and/or in the form of vegetative cells, spores, mycelia, conidia and/or any form of microbial propagule. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or medium resulting from the microbial growth can be removed from the growth vessel and transferred via, for example, piping for immediate use.

In other embodiments, the composition (microbes, medium, or microbes and medium) can be placed in containers of appropriate size, taking into consideration, for example, the intended use, the contemplated method of application, the size of the fermentation tank, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Upon harvesting the microbe-based composition from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, nutrients for plant growth, tracking agents, pesticides, herbicides, animal feed, food products and other ingredients specific for an intended use.

Advantageously, in accordance with the subject invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

The microbe-based products of the subject invention may be, for example, microbial inoculants, biopesticides, nutrient sources, remediation agents, health products, and/or bio surfactants.

In one embodiment, the fermentation products (e.g., microorganisms and/or metabolites) obtained after the cultivation process are typically of high commercial value. Those products containing microorganisms have enhanced nutrient content than those products deficient in the microorganisms. The microorganisms may be present in the cultivation system, the cultivation broth and/or cultivation biomass. The cultivation broth and/or biomass may be dried (e.g., spray-dried), to produce the products of interest.

In one embodiment, the cultivation products may be prepared as a spray-dried biomass product. The biomass may be separated by known methods, such as centrifugation, filtration, separation, decanting, a combination of separation and decanting, ultrafiltration or microfiltration. The biomass cultivation products may be further treated to facilitate rumen bypass. The biomass product may be separated from the cultivation medium, spray-dried, and optionally treated to modulate rumen bypass, and added to feed as a nutritional source.

In one embodiment, the cultivation products may be used as an animal feed or as food supplement for humans. The cultivation products may be rich in at least one or more of fats, fatty acids, lipids such as phospholipid, vitamins, essential amino acids, peptides, proteins, carbohydrates, sterols, enzymes, and trace minerals such as, iron, copper, zinc, manganese, cobalt, iodine, selenium, molybdenum, nickel, fluorine, vanadium, tin and silicon. The peptides may contain at least one essential amino acid.

In other embodiments, the essential amino acids are encapsulated inside a subject modified microorganism used in a cultivation reaction. The essential amino acids are contained in heterologous polypeptides expressed by the microorganism. Where desired, the heterologous peptides are expressed and stored in the inclusion bodies in a suitable microorganism (e.g., fungi).

In one embodiment, the cultivation products have a high nutritional content. As a result, a higher percentage of the cultivation products may be used in a complete animal feed. In one embodiment, the feed composition comprises the modified cultivation products ranging from 15% of the feed to 100% of the feed.

The subject invention further provides materials and methods for the production of biomass (e.g., viable cellular material), extracellular metabolites (e.g., both small and large molecules), and/or intracellular components (e.g., enzymes and other proteins). The microbes and microbial growth by-products of the subject invention can also be used for the transformation of a substrate, such as an ore, wherein the transformed substrate is the product.

The subject invention further provides microbe-based products, as well as uses for these products to achieve beneficial results in many settings including, for example, improved bioremediation, mining, and oil and gas production; waste disposal and treatment; enhanced health of livestock and other animals; and enhanced health and productivity of plants by applying one or more of the microbe-based products.

In specific embodiments, the systems of the subject invention provide science-based solutions that improve agricultural productivity by, for example, promoting crop vitality; enhancing crop yields; enhancing plant immune responses; enhancing insect, pest and disease resistance; controlling insects, nematodes, diseases and weeds; improving plant nutrition; improving the nutritional content of agricultural and forestry and pasture soils; and promoting improved and more efficient water use.

In one embodiment, the subject invention provides a method of improving plant health and/or increasing crop yield by applying the composition disclosed herein to soil, seed, or plant parts. In another embodiment, the subject invention provides a method of increasing crop or plant yield comprising multiple applications of the composition described herein.

Advantageously, the method can effectively control nematodes, and the corresponding diseases caused by pests while a yield increase is achieved and side effects and additional costs are avoided.

In another embodiment, the method for producing microbial growth by-products may further comprise steps of concentrating and purifying the by-product of interest.

In one embodiment, the subject invention further provides a composition comprising at least one type of microorganism and/or at least one microbial growth by-product produced by said microorganism. The microorganisms in the composition may be in an active or inactive form and/or in the form of vegetative cells, spores, mycelia, conidia and/or any form of microbial propagule. The composition may or may not comprise the growth matrix in which the microbes were grown. The composition may also be in a dried form or a liquid form.

In one embodiment, the composition is suitable for agriculture. For example, the composition can be used to treat soil, plants, and seeds. The composition may also be used as a pesticide.

In one embodiment, the subject invention further provides customizations to the materials and methods according to the local needs. For example, the method for cultivation of microorganisms may be used to grow those microorganisms located in the local soil or at a specific oil well or site of pollution. In specific embodiments, local soils may be used as the solid substrates in the cultivation method for providing a native growth environment. Advantageously, these microorganisms can be beneficial and more adaptable to local needs.

The cultivation method according to the subject invention not only substantially increases the yield of microbial products per unit of nutrient medium but also improves the simplicity of the production operation. Furthermore, the cultivation process can eliminate or reduce the need to concentrate microorganisms after finalizing fermentation.

Advantageously, the method does not require complicated equipment or high energy consumption, and thus reduces the capital and labor costs of producing microorganisms and their metabolites on a large scale.

Microbial Growth by-Products

The methods and systems of the subject invention can be used to produce useful microbial growth by-products such as, for example, biosurfactants, enzymes, acids, biopolymers, solvents, and/or other microbial metabolites. In specific embodiments, the growth by-product is a biosurfactant. Even more specifically, the growth by-product can be a biosurfactant selected from surfactin, sophorolipids (SLPs), rhamnolipids (RLPs) and mannosylerythritol lipids (MELs).

Biosurfactants are a structurally diverse group of surface-active substances produced by microorganisms. Biosurfactants are biodegradable and can be easily and cheaply produced using selected organisms on renewable substrates. Most biosurfactant-producing organisms produce biosurfactants in response to the presence of a hydrocarbon source (e.g., oils, sugar, glycerol, etc.) in the growing media. Other media components such as concentration of iron can also affect biosurfactant production significantly. For example, the production of RLPs by the bacteria Pseudomonas aeruginosa can be increased if nitrate is used as a source of nitrogen rather than ammonium. Also the concentration of iron, magnesium, sodium, and potassium; the carbon:phosphorus ratio; and agitation can greatly affect rhamnolipid production.

All biosurfactants are amphiphiles. They consist of two parts: a polar (hydrophilic) moiety and non-polar (hydrophobic) group. Due to their amphiphilic structure, biosurfactants increase the surface area of hydrophobic water-insoluble substances, increase the water bioavailability of such substances, and change the properties of bacterial cell surfaces.

Biosurfactants include low molecular weight glycolipids (e.g., rhamnolipids, sophorolipids, mannosylerythritol lipids), lipopeptides (e.g., surfactin), flavolipids, phospholipids, and high molecular weight polymers such as lipoproteins, lipopolysaccharide-protein complexes, and polysaccharide-protein-fatty acid complexes. The common lipophilic moiety of a biosurfactant molecule is the hydrocarbon chain of a fatty acid, whereas the hydrophilic part is formed by ester or alcohol groups of neutral lipids, by the carboxylate group of fatty acids or amino acids (or peptides), organic acid in the case of flavolipids, or, in the case of glycolipids, by the carbohydrate.

Microbial biosurfactants are produced by a variety of microorganisms such as bacteria, fungi, and yeasts. Exemplary biosurfactant-producing microorganisms include Pseudomonas species (P. aeruginosa, P. putida, P. florescens, P. fragi, P. syringae); Flavobacterium spp.; Bacillus spp. (B. subtilis, B. pumillus, B. cereus, B. licheniformis); Wickerhamomyces spp., Candida spp. (C. albicans, C. rugosa, C. tropicalis, C. lipolytica, C. torulopsis); Rhodococcus spp.; Arthrobacter spp.; campylobacter spp.; cornybacterium spp.; Pichia spp.; Starmerella spp.; and so on. The biosurfactants may be obtained by fermentation processes known in the art.

Other microbial strains including, for example, other fungal strains capable of accumulating significant amounts of glycolipid-biosurfactants, for example, and/or bacterial strains capable of accumulating significant amounts of, surfactin, for example, can be used in accordance with the subject invention. Other metabolites useful according to the present invention include mannoprotein, beta-glucan and other biochemicals that have bio-emulsifying and surface/interfacial tension-reducing properties.

In one embodiment of the subject invention, the biosurfactants produced by the subject systems include surfactin and glycolipids such as rhamnolipids (RLP), sophorolipids (SLP), trehalose lipids or mannosylerythritol lipids (MEL). In particular embodiments, the subject system is used to produce SLPs and/or MELs on a large scale.

Sophorolipids are glycolipid biosurfactants produced by, for example, various yeasts of the Starmerella clade. Among yeasts of the Starmerella clade that have been examined, the greatest yield of sophorolipids has been reported from Candida apicola and Starmerella bombicola. SLPs consist of a disaccharide sophorose linked to long chain hydroxy fatty acids. These SLPs are a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid is generally 16 or 18 carbon atoms, and may contain one or more unsaturated bonds. The fatty acid carboxyl group can be free (acidic or open form) or internally esterified at the 4″-position (lactone form).

Mannosylerythritol lipids are a glycolipid class of biosurfactants produced by a variety of yeast and fungal strains. Effective MEL production is limited primarily to the genus Pseudozyma, with significant variability among the MEL structures produced by each species. MELs contain 4-O-b-D-mannopyranosyl-erythritol as their sugar moiety or a hydrophilic unit. According to the degree of acetylation at C-4′ and C-6′ positions in mannopyranosyl, MELs are classified as MEL-A, MEL-B, MEL-C and MEL-D. MEL-A represents the diacetylated compound whereas MEL-B and MEL-C are monoacetylated at C-6′ and C-4′, respectively. The completely deacetylated structure is attributed to MEL-D. Outside of Pseudozyma, a recently isolated strain, Ustilago scitaminea, has been shown to exhibit abundant MEL-B production from sugarcane juice. MELs act as effective topical moisturizers and can repair damaged hair. Furthermore, these compounds have been shown to exhibit both protective and healing activities, to activate fibroblasts and papilla cells, and to act as natural antioxidants.

Due to the structure and composition of SLPs and MELs, these biosurfactants have excellent surface and interfacial tension reduction properties, as well as other beneficial biochemical properties, which can be useful in applications such as large scale industrial and agriculture uses, and in other fields, including but not limited to cosmetics, household products, and health, medical and pharmaceutical fields.

Biosurfactants accumulate at interfaces, thus reducing interfacial tension and leading to the formation of aggregated micellular structures in solution. Safe, effective microbial biosurfactants reduce the surface and interfacial tensions between the molecules of liquids, solids, and gases. The ability of biosurfactants to form pores and destabilize biological membranes permits their use as antibacterial, antifungal, and hemolytic agents. Combined with the characteristics of low toxicity and biodegradability, biosurfactants are advantageous for use in the oil and gas industry for a wide variety of petroleum industry applications, such as microbially enhanced oil recovery. These applications include, but are not limited to, enhancement of crude oil recovery from an oil-containing formation; stimulation of oil and gas wells (to improve the flow of oil into the well bore); removal of contaminants and/or obstructions such as paraffins, asphaltenes and scale from equipment such as rods, tubing, liners, tanks and pumps; prevention of the corrosion of oil and gas production and transportation equipment; reduction of H₂S concentration in crude oil and natural gas; reduction in viscosity of crude oil; upgradation of heavy crude oils and asphaltenes into lighter hydrocarbon fractions; cleaning of tanks, flowlines and pipelines; enhancing the mobility of oil during water flooding though selective and non-selective plugging; and fracturing fluids.

When used in oil and gas applications, the systems of the present invention can be used to lower the cost of microbial-based oilfield compositions and can be used in combination with other chemical enhancers, such as polymers, solvents, fracking sand and beads, emulsifiers, surfactants, and other materials known in the art.

Biosurfactants produced according to the subject invention can be used for other, non-oil recovery purposes including, for example, cleaning pipes, reactors, and other machinery or surfaces, as well as pest control, for example, when applied to plants and/or their surrounding environment. Some biosurfactants produced according to the subject invention can be used to control pests because they are able to penetrate through pests' tissues and are effective in low amounts without the use of adjuvants. It has been found that at concentrations above the critical micelle concentration, the biosurfactants are able to penetrate more effectively into treated objects.

Pests can be controlled using either the biosurfactant-producing organisms as a biocontrol agent or by the biosurfactants themselves. In addition, pest control can be achieved by the use of specific substrates to support the growth of biosurfactant-producing organisms as well as to produce biosurfactant pesticidal agents. Advantageously, natural biosurfactants are able to inhibit the growth of competing organisms and enhance the growth of the specific biosurfactant-producing organisms.

In addition, these biosurfactants can play important roles in treating animal and human diseases. Animals can be treated by, for example, by dipping or bathing in a biosurfactant solution alone, with or without microbe cell mass, and/or in the presence of other compounds such as copper or zinc.

The compositions produced according to the present invention have advantages over biosurfactants alone due to the use of entire cell culture, including: high concentrations of mannoprotein as a part of yeast cell wall's outer surface (mannoprotein is a highly effective bioemulsifier capable of reaching up to an 80% emulsification index); the presence of the biopolymer beta-glucan (an emulsifier) in yeast cell walls; the presence of sophorolipids in the culture, which is a powerful biosurfactant capable of reducing both surface and interfacial tension; and the presence of metabolites (e.g., lactic acid, ethanol, etc.) in the culture. These compositions can, among many other uses, act as biosurfactants and can have surface/interfacial tension-reducing properties.

Cultivation of microbial biosurfactants according to the prior art is a complex, time and resource consuming, process that requires multiple stages. The subject invention provides equipment, apparatuses, methods and systems that simplify and reduce the cost of this process. The subject invention also provides novel compositions and uses of these compositions.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1 Multi-Tank Fermentation System

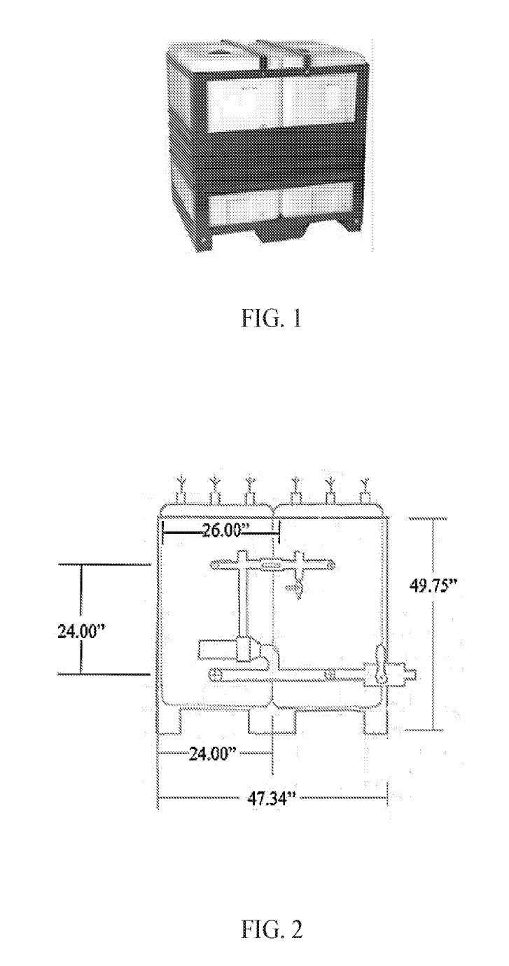

A portable and distributable plastic reactor was constructed as shown in FIGS. 1 and 2. The reactor has two plastic square tanks with two loops for mass exchange between the two tanks.

The top of the system was equipped with a pumping mechanism to pull from a first tank and deposit in a second tank, which accounts for one of the loops. The other loop was at the bottom of the tank and relied on hydrostatic pressure to equalize the volumes in the tanks.

The addition of filtered air into the tanks was controlled by a sparging mechanism that ran through a bubbler. The filtered air for sparging was generated via a high volume aquatic pumping system. There were two 72 inch bubblers per tank, resulting in a total of four per system. An air compressor was also used to add filtered air into the top and bottom loops for extra aeration.

The top loop was equipped with a sight glass to allow for viewing the culture's turbidity, color, thickness and other characteristics. The reactor had a working volume of 750-850 L for growing Starmerella yeast for cell and metabolite production (however, size and scale can vary depending on the required application). The reactor is particularly well-suited for mass production of Starmerella clade yeast on small or large scales.

In order to further reduce the cost of culture production and ensure scalability of the technology, the system was not sterilized using traditional methods. Instead, a method of empty vessel sanitation was used that included treatment of internal surfaces with 2-3% hydrogen peroxide and rinsing with bleach and high pressure hot water. Additionally, in order to reduce the possibility of contamination, water used for preparing the culture was filtered through a 0.1-micron filter.

The culture medium components were temperature decontaminated at 85-90° C. or dissolved in 3% hydrogen peroxide (dry components and H₂O ratio is 1:3 v/v), except for the oil, which was only temperature decontaminated.

The fermentation temperature should generally be between about 23 to 37° C., and preferably between about 25 to 30° C.

The pH should be from about 3 to 5, and preferably between about 3.5 to about 4.5. Additionally, in order to further reduce the possibility of contamination, the cultivation process began at a pH of 4.0-4.5 and then was further conducted at an average pH of 3.2-3.5.

Under these cultivation conditions, industrially useful production of biomass, sophorolipids and other metabolites were achieved from about 60 to about 120 hours of fermentation. Upon completion of the fermentation, the culture can then be applied for a variety of industrial purposes.

### Example 2 Culture Medium and its Use for Starmerella Cultivation in Multi-Tank Reactor

A medium composed of 20-100 gL⁻¹ glucose, 0-50 gL⁻¹ (which can change, e.g., depending on the desired amount of biosurfactant to be produced) canola oil, 5 gL⁻¹ yeast extract, 4 gL⁻¹ NH₄Cl, 1 gL⁻¹ KH₂PO₄.H₂O, 0.1 gL⁻¹ NaCl and 0.5 gL⁻¹ MgSO₄.7H₂O, was prepared in filtered water.

The initial pH was adjusted to about 4.5 with 6N KOH. The cultures were grown at about 25° C. The cultivation times were up to 120 h and the pH of the reactor cultures were adjusted to about 3.5 twice daily by the addition of 1.0M NaOH.

At these cultivation conditions, the amount of Starmerella wet biomass reached up to 100 grams per liter of culture.

### Example 3—Seed Culture Preparation Using Antibiotics

The following is one example of a method for preparing scaled up microbe-based products according to the subject invention. A seed culture can be prepared and then scaled up for use in the subject systems. Scaling up can occur in a separate vessel, for example, by adding the reagents to a drum mixer, and allowing the culture to grow for 2 or more days. After the seed culture has been allowed to grow for at least 2 days in the mixer, the culture can be divided into an appropriate number of portions for inoculating any number of the subject reactor systems.

### Media Composition

| Reagent | Weight (g/L) |
|---|---|
| Yeast Extract | 5 |
| Glucose | 100 |
| Urea | 1 |
| Streptomycin (Antibiotic) | 0.1 |
| Oxytetracycline (Antibiotic) | 0.01 |

### Shake Flasks

Two liters of the media composition were prepared without the antibiotics in 4 L flasks. The flasks were then prepped for autoclaving. A piece of cheese cloth, followed by a piece of blue autoclave paper, was secured to the rim of the flasks using a rubber band. (The cheese cloth and blue paper must be large enough so that the cloth and paper extend beyond where the rubber band secures the pieces to the rim.) Autoclave cycles occurred at 121° C. for 20 minutes, then the flasks were allowed to cool down to 30° C. or lower.

Next, the antibiotics were weighed out and dissolved with DI water in a beaker or a 50 mL conical tube. Agar plates were labeled with C. bombicola or S. bombicola. Single colonies were selected from the plate with a loop (one to two loops should be sufficient), practicing aseptic technique, and the flasks were inoculated under the hood in the lab. The dissolved antibiotics were also added to the flasks. When removing and replacing the cheese cloth and autoclave blue paper under the hood, care was taken so as not to touch the bottom of the cheese cloth that was exposed to the inside of the flask.

Once the 4 L flasks were inoculated, they were placed in shakers in a fermentation room. The temperature of the shakers was set to 30° C. The flasks were allowed to ferment for at least 2 days before use of the seed culture. Samples of the seed culture inoculum were taken under a hood before use to ensure the inoculum was pure and without contamination. Slides of the samples were made using simple gram stain.

### Scaling Up Using Drum Mixer

After the seed culture was allowed to grow for 2 days, the seed culture was scaled up in a black drum mixer for inoculating the reactors. Dry ingredients for a 40 L batch of the reagents listed above were weighed out. Antibiotics were weighed out and kept in a separate container.

The media components were dissolved in a 40 L carboy, ensuring that the volume did not exceed that 40 L level. The 40 L of media were added to the mixer, followed by 2 L of inoculum from the shakers and the appropriate amount of dissolved antibiotics. The culture was then allowed to grow for at least 2 days in the mixer before portions were transferred out for the reactors. The amount of culture portioned into each cubicle depended on how many liters of culture were produced and the number of cubicles to be started. Each reactor was given at least 10 L of culture.

The scaled up culture was harvested using a drum pump in either 20 L or 40 L carboys, depending on how much volume of culture was needed per reactor. The culture was then transferred out of the carboys with the same drum pump and the reactors were inoculated.

### Cleaning the Drum Mixer

After harvesting all the culture out of the drum mixer, the mixer was moved to a drain rinsed with warm water, taking care to remove any biofilm. After thorough rinsing, 70% IPA was used to sterilize the reactor. The mixer was allowed to dry, and when no IPA residual was left over, another seed culture batch could begin.

### Example 3—Operation of the Multi-Tank Reactor Using a Culture Medium Comprising Antibiotics

### Culture Media

| Reagent | Weight (g/L) |
|---|---|
| Urea | 1 |
| Yeast Extract | 5 |
| Glucose | 60 |
| Canola Oil | 70 ml/L |
| Streptomycin (Antibiotic) | 0.1 |
| Oxytetracycline (Antibiotic) | 0.01 |

### Prepping the Multi-Tank Reactor

The total volume of the two-tank reactor was 750 L, so the appropriate amount of the reagents above were determined and weighed out. Dry ingredients were dissolved in a barrel using filtered water. Canola oil was not added during the dissolving step. Antibiotics were kept separate, and dissolved in DI water in a large beaker.

Next the dissolved media were added to the reactor. The reactor was filled up to ˜185 gallons total with filtered water, followed by the canola oil. The final volume was 100 gallons in each tank, thus equaling 200 gallons total.

The starting temperature was at least 23° C. but no higher than 30° C. Once temperature was established in the range of 23 to 30° C., inoculum was added from the mixer, followed by the dissolved antibiotics. Samples were taken to measure pH and DO %. The culture was allowed to grow for a minimum of ˜3 days, monitoring pH, temperature, and DO % at least once each day. Once the cube was ready for harvesting, a sample was taken to measure pH.

A slide was made, a serial diluted plate was made, and an OD measurement was performed for quality control/assurance. DO and temperature were also measured. Once quality of the culture was assured, the culture was harvested by using the camlock fitting at the bottom loop and a transfer pump equipped with two sets of hoses.

In the event that the quality of the culture batch was unfit for use, half a bottle of bleach was poured between the two tanks of the reactor, allowed to sit for 20 minutes, and then drained.

### Cleaning the Reactor

First, the reactor tanks were rinsed out. The reactor was unplugged from the wall, both bottom ball valves were shut off, and the bottom loop assembly was removed by disconnecting the camlock fittings. Care was taken not to let too much media spill when taking off the bottom loop. With the loop removed, a palate jack was used to transfer the reactor toward a drain in the warehouse.

The top loop was then removed and the bottom loop components were rinsed with hot water. If these loops or their components were overly dirty, a disinfectant was used to clean them thoroughly. Next, the tanks were rinsed out using hot water and the spray nozzle on the hose in the warehouse close to the drain. Care was taken to remove any film inside the tanks.

Next, the inside of the tanks were fogged with 3% hydrogen peroxide (H₂O₂) for at least 3-5 minutes. Wearing PPE was crucial for this step.

Note: If contamination was of concern, the reactor was transferred back to its running position and the tanks filled up with 0.5% hydrogen peroxide solution. Total volume of the reactor was about 1100 L, so about 55 L of 10% hydrogen peroxide was needed. The bottom loop was reassembled and the unit turned on. The reactor was allowed at least 4 hours to thoroughly clean itself with the 0.5% hydrogen peroxide. Once at least 2 hours had elapsed the unit was turned off. The bottom loop ball valves were shut off, and the bottom loop disconnected. A palate jack was used to transfer the unit over to the drain for draining.

Then the reactor was rinsed thoroughly with filtered (preferably hot) water, and was ready for another batch to begin.

### Example 4—Paraffin Liquefaction

An experiment was conducted to show the efficacy of a Starmerella culture on paraffin liquefaction. The results of the experiment can be seen in FIG. 2, and the results of a culture are marked as “Star3.”

Twenty-one (21) microbial and chemical emulsification products (including commercial) were investigated for paraffin degradation efficacy. Fifty (50) mL Falcon tubes with a working volume of 25 mL were used in the experiment. Solid paraffin was obtained from an oilfield. Four (4.0) grams of solid paraffin was weighed and then added into each Falcon tube and 20 mL of each liquid from Table 1 was added to the Falcon tubes. All the Falcon tubes were then horizontally placed in an ENVIRO GENE incubator at 30° C. to 40° C. and gently mixed. After different incubation times (1, 2, or 4 days), the tubes were collected and analyzed.

Three (3) sets of experiments were carried out at different incubation times and different temperatures. The first set of experiments was performed at 30° C. In this set of experiments, “Star3” contained S. bombicola with around 4 g/L sophorolipid (which is roughly the saturation level). The “Star3” treatment showed complete spreading within the tubes, and was the only additive to completely turn the paraffin into liquid, whereas paraffin maintained solid forma in all other tests (including commercial Naxan). This proof of concept experiment showed that a Starmerella culture can be highly effective for liquefaction of paraffin, and even superior to other commercially available chemicals and biologicals.

1. A system for producing a microbe-based composition comprising:
a reactor comprising a first tank and a second tank;
a pump having an input connected to the first tank via a first tube, and an output connected to the second tank via a second tube;
a third tube connecting from the second tank to the first tank wherein said third tube is suitable for allowing liquid to flow under hydrostatic pressure from the second tank to the first tank.

2. The system of claim 1, further comprising one or more air blowers or air compressors, wherein the one or more air blowers or compressors are connected to one or more gas injectors, bubblers, and/or spargers.

3. The system of claim 1, wherein the reactor has a working volume of 10 to 40,000 gallons.

4. The system of claim 1, further comprising a frame for supporting system components.

5. The system of claim 1, further comprising wheels and handles for maneuvering the system.

6. The system of claim 1, wherein the system is configured on the back of a truck trailer and/or semi-trailer, and/or wherein the system is portable.

7. The system of claim 1, wherein the one or more pumps are capable of establishing a recycle ratio ranging from 30 to 0.1.

8. A method for cultivating microorganism without contamination, wherein said method comprises:
adding a culture medium comprising water and nutrient components to the system of claim 1 using a peristaltic pump;
inoculating the system with a viable microorganism; and
optionally, adding an antimicrobial agent to the system.

9. The method of claim 8, wherein the microorganism is a yeast.

10. The method of claim 9, wherein the microorganism is Starmerella bombicola.

11. The method of claim 9, wherein the microorganism is Pseudozyma aphidis.

12. The method of claim 8, wherein the system of claim 1 is sterilized prior to cultivating the microorganism.

13. The method of claim 12, wherein sterilization comprises:
washing the internal surfaces of the reactor with a disinfectant;
fogging the inside of the reactor with a 3% hydrogen peroxide solution; and/or
steaming the inside of the reactor with water at a temperature of 105° C. to 110° C.

14. The method of claim 8, wherein the culture medium is decontaminated prior to being added to the system.

15. The method of claim 14, wherein decontamination is achieved by:
autoclaving the culture medium components;
filtering the water using a 0.1-micron water filter; and
UV sterilizing the water.

16. (canceled)

17. The method of claim 8, wherein the antimicrobial agent is biosurfactant.

18. A composition comprising a microorganism and/or one or more products of the growth of that microorganism produced by the system of claim 1.

19. (canceled)

20. The composition of claim 18, wherein the microorganism is Starmerella bombicola.

21. The composition of claim 18, wherein the microorganism is Pseudozyma aphidis.

22. The composition of claim 18, wherein the growth by-product is a biosurfactant.

23. The composition of claim 22, wherein the biosurfactant is a sophorolipid.

24. The composition of claim 22, wherein the biosurfactant is a mannosylerythritol lipid.

25. A method for enhancing the amount of oil recoverable from an oil-containing formation, wherein said method comprises applying a composition of claim 18 to the oil-containing formation.

26. A method for cleaning an oil well rod, tubing and/or casing, wherein said method comprises applying to the oil well rod, tubing and casing structures a composition of claim 18.

27. A method for improving plant growth, yield, and/or health, wherein said method comprises applying to the plant or its environment a composition of claim 18.

28. A method for controlling a pest of animals wherein said method comprises contacting the pest with a composition of claim 18.

29. A method for feeding an animal, wherein the method comprises adding the composition of claim 18 to the animal's food and/or drinking water source.
